SEQUENCE LISTING (2 sequences)

SEQ ID NO: 1 - RNA, 22 nt, Artificial Sequence
  miRNA-214-3p mimic
ugccugucuacacuugcugugc

SEQ ID NO: 2 - RNA, 22 nt, Artificial Sequence
  miRNA-214-3p inhibitor
acagcaggcacagacaggcagu

(19) United States
(12) Patent Application Publication
(10) Pub. No.: US 2025/0002909 A1
(43) Pub. Date: Jan. 2, 2025

(54) COMPOSITION FOR PREVENTING OR TREATING NEURODEGENERATIVE DISEASES CONTAINING A miRNA INHIBITOR AND USE THEREOF

(71) Applicant: IUCF-HYU (INDUSTRY-UNIVERSITY COOPERATION FOUNDATION HANYANG UNIVERSITY), Seoul (KR)

(72) Inventors: Seung Hyun KIM, Seoul (KR); Min Young NOH, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 18/290,373

(22) PCT Filed: May 13, 2022
(86) PCT No.: PCT/KR2022/006914

§ 371 Date: Nov. 13, 2023

(30) Foreign Application Priority Data
May 14, 2021 (KR) 10-2021-0062983

(51) Int. Cl. C12N 15/113; A61P 25/28

(52) U.S. Cl. CPC C12N 15/113; A61P 25/28; C12N 2310/113

(57) ABSTRACT

The present invention relates to a pharmaceutical composition, for preventing or treating a neurodegenerative disease, comprising a miRNA-214 inhibitor capable of controlling expression of miRNA-214 or a gene to be bound thereto. The pharmaceutical composition comprising a miRNA-214 inhibitor exhibits effects such as phagocytic function recovery, reduced neuroinflammation factors in microglia, autophagy-related factor control, expression/activation of nucleocytoplasmic trafficking-related factors and the like, and thus may be used for preventing and treating a neurodegenerative disease.

### TECHNICAL FIELD

The present disclosure relates to a composition for preventing or treating neurodegenerative diseases, the composition containing a miRNA-214 inhibitor capable of regulating the expression of the miRNA-214 or a binding partner gene to the miRNA-214.

### BACKGROUND ART

Neurodegenerative diseases are diseases that cause abnormalities in motor control ability, cognitive function, perceptual function, sensory function, and autonomic nerve function due to a loss of nerve structure and function. As the population rapidly ages around the world, the number of patients with neurodegenerative diseases such as Amyotrophic Lateral Sclerosis (ALS), Alzheimer's disease (AD), and Parkinson's disease (PD) is rapidly increasing. These neurodegenerative diseases often leave sequela since nerve cells have the characteristics of having difficulties in repairment and regeneration once damaged or injured; thereby, patients do not return to normal, and the diseases continue to worsen. Therefore, research has been actively conducted worldwide for decades to solve these problems, but the exact cause of the diseases has not been properly identified. Because of these characteristics, most neurodegenerative diseases cannot be cured with current treatment techniques, and the focus is mainly on targeting neuroinflammation and related pathways to regulate the progression rate and alleviate symptoms of the diseases. Therefore, there is an urgent need to develop new treatment targets and treatments for neurodegenerative diseases.

The main pathological mechanisms of neurodegenerative diseases are largely as follows: 1) abnormal protein aggregation, 2) abnormal RNA metabolism and transport, 3) neuroinflammation, 4) apoptosis, and 5) oxidative stress.

The abnormal protein aggregation refers to a condition in which abnormal insoluble aggregates formed of major protein molecules causing neurodegenerative diseases (for example, SOD1, Amyloid beta, FUS, TDP-43, and alpha-synuclein) cause cytotoxicity of nerve cells, resulting in dysfunction and apoptosis of the nerve cells. In addition, the abnormal RNA metabolism is caused by the formation of RNA-protein aggregates. The abnormal RNA-protein interaction causes the impairment of assembly/disassembly dynamics of stress granules (SG) as membrane-less structures. These irreversible RNA-protein dynamics are caused by genetic or environmental factors, leading to the formation of pathogenic aggregates within nerve cells and, ultimately, resulting in neural cell death. In addition to ALS causative abnormal aggregated proteins, a recent study revealed that Tau and alpha-synuclein, which are also important components of SG, can be transformed into aggregated proteins in AD and PD, respectively, when these proteins are changed by mutation of the corresponding genes or hyperphosphorylation of the proteins. Aggregated hyper-phosphorylated Tau and misfolded alpha-synuclein changes the SG dynamics and forms the pathogenic aggregates in neuronal cells. Therefore, there is growing interest in the possibility of treating neurodegenerative diseases through the regulation of NCT (Nucleocytoplasmic transport), which controls the exchange of proteins and RNA between the nucleus and the cytoplasm, and the role of a disaggregase to rescue the irreversible pathological changes to normal status, and the activation of autophagic function.

Neuroinflammation is a part of the immune system's protective response to tissue damage or microbial invasion, and these functions are well-regulated under normal conditions. However, under neurodegenerative disease conditions, microglia are aberrantly activated by various unfolded or aggregated proteins (for example, amyloid-β in AD, α-synuclein in PD, SOD1 or FUS in ALS, TDP-43 in frontotemporal dementia [FTD]). Not only aberrant activation, but the functional changes of the microglial cells act as a stimulator of the pro-inflammatory response, causing pathological neuroinflammation, and finally contribute to the propagation and progression of neurodegenerative changes in ALS, AD, and PD. Therefore, dysfunction of microglia, including the defective phagocytic function and secretion of pro-inflammatory cytokines has been empathized in understating the pathophysiological mechanism of neuroinflammation in neurodegenerative diseases. For this reason, interest in the development of therapeutics that regulate the function of microglia is increasing.

MicroRNAs (miRNAs) are small-RNA molecules consisting of about 22 nucleotides and regulate gene expression through the destruction of target mRNA or inhibition of the target mRNA at the translation stage. The miRNAs are involved in various physiological conditions and diseases. In the central nervous system, deletion of Dicer, which is a key regulator of miRNA production, induces impairment of neurogenesis, showing that balanced expression of the miRNAs plays an important role in the development and degeneration of the nervous system. The miRNAs are detectable at stable levels in the blood and other body fluids and can be used as a tool for disease diagnosis or predicting the prognosis. To date, many studies have reported that the level of miRNAs is differentially expressed in body fluids, including cerebrospinal fluid (CSF) and plasma, when compared to patients with neurodegenerative diseases and healthy controls. The possibility that miRNA could be a therapeutic target in neurodegenerative diseases has already been emphasized, but specific treatment strategies regulating miRNA, though still modest, have begun to be attempted only recently.

Therefore, the present disclosure seeks to develop a use for preventing or treating neurodegenerative diseases using a miRNA inhibitor, specifically focusing on miRNA-214, capable of modulating the impaired phagocytic function of microglia as one of the key pathophysiological mechanisms of neurodegenerative diseases.

### DISCLOSURE

### Technical Problem

Against the background, the present inventors have made efforts to discover methods for preventing and treating neurodegenerative diseases using an inhibitor to regulate biomarkers related to neurodegenerative diseases. As a result, the inventors confirmed that the miRNA-214 of the present disclosure could predict the prognosis of neurodegenerative diseases through the evidence of miRNA-214-3p regulating the expression of genes such as modulating microglial phagocytic capacity (NCKAP1, WASF2, and ABL2), key factors in the neuroinflammatory mechanism of microglia (TRAF1, TNFSF10, and IKBKB), autophagy-related factors (RUBCN, ATG16L1, and ATG12), and Nucleocytoplasmic Trafficking-related factors (TNPO1, IPO11, and KPNA1), and the inventors completed the present disclosure.

One aspect is to provide a pharmaceutical composition for preventing or treating neurodegenerative diseases, the pharmaceutical composition containing a miRNA-214-3p inhibitor as an active ingredient.

Another aspect is to provide a pharmaceutical agent for preventing or treating neurodegenerative diseases containing the miRNA-214-3p inhibitor as an active ingredient.

Another aspect is to provide a method of preventing or treating a neurodegenerative disease, the method including administering a pharmaceutical composition containing the miRNA-214-3p inhibitor to a subject.

However, the technical problems to be achieved by the present disclosure are not limited to the problems mentioned above, and other problems not mentioned will be clearly understood by those skilled in the art from the description below.

### Technical Solution

The present inventors have confirmed that a miRNA-214-3p inhibitor capable of regulating the expression of the miRNA-214-3p and the expression of the binding partner of the miRNA-214-3p could be used for preventing or treating neurodegenerative diseases, and the inventors completed this disclosure.

The present disclosure provides a pharmaceutical composition for preventing or treating neurodegenerative diseases, the pharmaceutical composition containing the miRNA-214-3p inhibitor as an active ingredient.

The term “miRNA-214-3p inhibitor” is used to generally refer to all agents that reduce the expression or activity of miR-214-3p. Specifically, the miRNA-214-3p inhibitor includes all the agents that reduce the expression level of miRNA-214-3p at the transcription stage, mRNA level, or translation level, or that reduce the activity of miRNA-214-3p by interfering with the activity of miRNA-214-3p in ways that the inhibitor affects the reduction of the expression of the miRNA-214-3p, acts directly on the miRNA-214-3p, or indirectly acts with a ligand or binding partner of the miRNA-214-3p.

In one embodiment of the present disclosure, the inhibitor may be an activity inhibitor or expression inhibitor against the miRNA-214-3p or an activity inhibitor or expression inhibitor against a binding partner of the miRNA-214-3p.

The miRNA-214-3p inhibitor can be used without limitation in their form, such as a compound, a nucleic acid, a peptide, a peptide mimic, a substrate analog, an aptamer, an antibody, a virus, or a vector containing the nucleic acid which can inhibit the expression or activity of the miRNA-214-3p or the expression or activity of the binding partner of the miRNA-214-3p through targeting.

In one embodiment of the present disclosure, the miRNA-214-3p inhibitor may be at least one selected from the group consisting of siRNA or shRNA (that decomposes the miRNA-214-3p gene or the mRNA being a binding partner of the miRNA-214-3p), an antisense oligonucleotide, RNAi, siRNA, miRNA, shRNA, and ribozyme (that reduce expression of the miRNA-214-3p).

In one embodiment of the present disclosure, the miRNA-214-3p inhibitor may be an aptamer or a small-molecular compound that binds to the target binding protein of the miRNA-214-3p and inhibits the function of the miRNA-214-3p. In addition, the miRNA-214-3p inhibitor is not limited to the mentioned above but may include siRNA or shRNA that decomposes the mRNA of the gene being a binding partner to the miRNA-214-3p and may include an antisense oligonucleotide that reduces the expression of the target binding protein of the miRNA-214-3p. In addition, the miRNA-214-3p inhibitor may be an aptamer or a small-molecular compound acting as the miRNA-214-3p inhibitor that binds to the target binding protein of the miRNA-214-3p and inhibits the function of the miRNA-214-3p.

In one embodiment of the present disclosure, the miRNA-214-3p inhibitor acts on phagocytosis caused by regulating the expression of mRNAs such as NCKAP1, WASF2, and ABL2 which are a binding partner of the miRNA-214-3p, thereby the inhibitor enables to show the preventive or therapeutic effect of neurodegenerative diseases.

The term “NCKAP1 (Nck-associated protein 1)”, as used herein, is a modulator of microglial phagocytic capacity, and the dysfunction of NCKAP1 can lead to neurodegenerative diseases.

The term “WASF2 (Wiskott-Aldrich syndrome protein family member 2)”, as used herein, is a protein encoded by the WASF2 gene. The WASF2 gene encodes a member of the Wiskott-Aldrich syndrome protein family, and dysfunction of the WASF2 gene can lead to neurodegenerative diseases.

The term “ABL2”, as used herein, is also known as Abelson-related gene (Arg) and one of the tyrosine protein kinases. The dysfunction of ABL2 can lead to neurodegenerative diseases.

In one embodiment of the present disclosure, the miRNA-214-3p inhibitor acting neuroinflammation caused by regulating mRNA expression of TRAF1, TRAF 3, TRAF 5, TRAF 7, TBFSF10, CD27, and IKBKB which are the binding partner of the miRNA-214-3p, thereby the inhibitor enables to show the preventive or therapeutic effect of neurodegenerative diseases.

The terms “TRAF1 (TNF Receptor Associated Factor 1)”, “TRAF3 (TNF Receptor Associated Factor 3)”, “TRAF5 (TNF Receptor Associated Factor 5)” and “TRAF7 (TNF Receptor Associated Factor 7)”, as used herein, are TNF receptor-related factors and are included into a group of proteins primarily involved in the regulation of inflammation, antiviral responses, and apoptosis. The TRAF proteins act on the mechanism of neuroinflammation and apoptosis, and the dysfunction of the TRAF proteins can lead to neurodegenerative diseases.

The term “TNFSF10 (TNF Superfamily Member 10)”, as used herein, is a TNF-related apoptosis-inducing ligand (TRAIL) and a protein that functions as a ligand that induces the apoptosis process. The TNFSF10 protein acts on the neuroinflammation mechanism, and the dysfunction of the TNFSF10 protein can lead to neurodegenerative diseases.

The term “CD27”, as used herein, refers to one member of the tumor necrosis factor receptor superfamily and a costimulatory immune checkpoint molecule. The CD27 protein acts on the mechanism of neuroinflammation and apoptosis, and the dysfunction of the CD27 protein can lead to neurodegenerative diseases.

The term “IKBKB (Inhibitor of Nuclear Factor Kappa B Kinase Subunit Beta)”, as used herein, acts on the mechanism of neuroinflammation and apoptosis, and the dysfunction of IKBKB can lead to neurodegenerative diseases.

In one embodiment of the present disclosure, the miRNA-214-3p inhibitor acts on autophagy caused by regulating mRNA expression of RUBCN, ATG16L1, ATG13, ATG12, MAPK1 which are binding partners of the miRNA-214-3p, and the inhibitor enables to show the preventive or therapeutic effect of neurodegenerative diseases.

The term “RUBCN (Rubicon Autophagy Regulator)”, as used herein, acts on autophagy, and the dysfunction of RUBCN can lead to neurodegenerative diseases.

The term “ATG16L1 (Autophagy Related 16 Like 1)”, as used herein, acts on LC3 lipidation and autophagosome formation, as well as on autophagy, and the dysfunction of ATG16L1 can lead to neurodegenerative diseases.

The terms “ATG12 (Autophagy Related 12)” and “ATG13 (Autophagy Related 13)”, as used herein, act on autophagy, and the dysfunction of ATG12 can lead to neurodegenerative diseases.

The term “MAPK1 (Mitogen-Activated Protein Kinase 1)”, as used herein, acts on the MAP kinase signaling pathway, as well as on autophagy, and the dysfunction of MAPK1 can lead to neurodegenerative diseases.

In one embodiment of the present disclosure, the miRNA-214-3p inhibitor acts on RNA transport caused by regulating the expression of mRNAs such as “TNPO1 (Transportin 1)”, “IPO11 (Importin 11)”, “KPNA1 (Karyopherin Subunit Alpha 1)” which are a binding partner of the miRNA-214-3p, thereby the inhibitor enables to show the preventive or therapeutic effect of neurodegenerative diseases.

In one embodiment of the present disclosure, the miRNA-214-3p inhibitor acts on apoptosis caused by regulating the expression of mRNAs such as BAX, TRAF1, TRAF 3, TRAF 5, TRAF 7, TBFSF10, CD27, and IKBKB which are a binding partner of the miRNA-214-3p, thereby the inhibitor enables to show the preventive or therapeutic effect of neurodegenerative diseases.

The term “BAX (BCL2 Associated X, Apoptosis Regulator)”, as used herein, is involved in the death of nerve cells during development, maintenance of homeostasis in the lymphatic system and reproductive organs, tumor suppression, cell death following DNA damage, and ischemia-reperfusion damage. The BAX protein acts on the mechanism of apoptosis, and the dysfunction of the BAX protein can lead to neurodegenerative diseases.

The term “TRAF10 (TNF Receptor Associated Factor 10)”, as used herein, is a TNF receptor-related factor and is included in a group of proteins mainly involved in the regulation of inflammation, antiviral response, and apoptosis. The TRAF10 protein acts on the mechanism of apoptosis, and the dysfunction of the TRAF10 protein can lead to neurodegenerative diseases.

In one embodiment of the present disclosure, the miRNA-214-3p inhibitor may be represented by at least one nucleic sequence selected from the group consisting of SEQ ID NO: 1 and SEQ ID NO: 3. At this time, the miRNA-214-3p inhibitor may be represented by a nucleic sequence with sequence homology of at least 70%, preferably at least 80%, more preferably at least 90%, and most preferably at least 95% to the nucleic sequences represented by SEQ ID NO: 1 or SEQ ID NO: 3.

In one embodiment, the miRNA-214-3p inhibitor may be represented by at least one nucleic sequence selected from the group consisting of the nucleic sequences represented by SEQ ID NO: 3.

The term “neurodegenerative disease”, as used herein, refers to Parkinson's disease, dementia, Alzheimer's disease, frontotemporal dementia, Huntington's disease, stroke, cerebral infarction, Pick's disease, head trauma, spinal cord injury, cerebral arteriosclerosis, ALS, multiple sclerosis, geriatric depression, or Creutzfeldt-Jakob disease. The term preferably refers to Parkinson's disease, dementia, Alzheimer's disease, frontotemporal dementia, Huntington's disease, or ALS (Amyotrophic lateral sclerosis), and more preferably ALS or Alzheimer's disease but is not limited to the mentioned above.

The term “prevention”, as used herein, refers to all actions that suppress or delay the onset of neurodegenerative diseases by administering the pharmaceutical composition according to the present disclosure.

The term “treatment”, as used herein, refers to all actions, leading to a situation where the symptoms of neurodegenerative diseases are improved or beneficially changed by administrating the pharmaceutical composition according to the present disclosure.

The composition according to the present disclosure can be used alone or in combination with surgery, radiation therapy, chemotherapy, and biological response regulators for preventing or treating neurodegenerative diseases, and the composition can be preferably used in combination with drugs that promote differentiation of nerve cells.

The composition according to the disclosure may further include a pharmaceutically acceptable carrier. The pharmaceutically acceptable carrier is commonly used in preparation and includes, but is not limited to, saline solution, sterilized water, IV drip, buffered saline solution, cyclodextrin, dextrose solution, maltodextrin solution, glycerol, ethanol, and liposome. If necessary, the carrier may further include other common additives such as antioxidants and buffers. In addition, diluents, dispersants, surfactants, binders, and lubricants may be additionally added to form injectable formulations such as aqueous solutions, suspensions, and emulsions; to form injections such as injection bags; and to form sprays such as aerosol formulations, pills, capsules, granules, or tablets. Regarding suitable pharmaceutically acceptable carriers and formulations, the formulations can be preferably carried out considering each ingredient by using the method disclosed in Remington's literature. The pharmaceutical preparation of the present disclosure is not limited to particular dosage forms but can be formulated such as in an injectable preparation, an infusion preparation, a spray formulation, a liquid formulation, or a topical skin preparation.

The composition can be administered orally or parenterally (for example, in areas intravenously, subcutaneously, intraperitoneally, or topically, the areas including the eye) depending on the preferred method. The dosage varies depending on patient's condition and weight, degree of disease, drug form, and administration route and time, but the dosage can be appropriately selected by those skilled in the art.

The composition can be administered in a pharmaceutically effective amount. The term “pharmaceutically effective amount”, as used herein, refers to an amount sufficient to treat or diagnose a disease in accordance with a reasonable benefit/risk ratio and refers to an amount applicable for medical treatment or diagnosis. The effective dose level can be determined based on factors including the type and severity of patient's disease, drug activity, sensitivity to the drug, administration time, administration route and excretion rate, treatment period, concurrently used drugs, and other factors well-known in the medical field. The composition may be administered as a single therapeutic agent or in combination with other therapeutic agents, may be administered sequentially or simultaneously with conventional therapeutic agents, and may be administered singly or multiple times. Considering all of the factors, it is important to administer an amount that can achieve maximum effect with the minimum amount without side effects, and this amount can be easily determined by those skilled in the art.

Specifically, the effective amount of the composition may vary depending on patient's age, gender, condition, weight, the absorption level of the active components in the body, inactivation rate and excretion rate, type of disease, and drugs used together. In general, the composition can be administered in an amount of 0.001 mg to 150 mg per kg of body weight, preferably 0.01 mg to 100 mg every day or every other day, or the administration can be divided into 1 to 3 times per day. However, the amount may increase or decrease depending on the administration route, severity of obesity, gender, weight, and age. Thus, the dosage does not limit the scope of the present disclosure in any way.

In addition, the present disclosure provides a health functional food composition for preventing or improving neurodegenerative diseases, the health functional food composition containing a miRNA-214-3p inhibitor as an active ingredient.

The term “improvement” may refer to a parameter related to the state of patients being treated. For example, the term refers to all actions that at least reduce the severity of symptoms. At this time, the health functional food can be used simultaneously or separately with drugs for treatment before or after the onset of the diseases for preventing or improving neurodegenerative diseases.

The active ingredients contained in the health functional food can be added directly to the food or used together with other foods or food ingredients, and the active ingredients can be used appropriately according to conventional methods. The amount of the mixed active ingredients can be appropriately determined depending on the purpose of use (for prevention or improvement). In general, when manufacturing the health functional food in the form of a food or beverage, the health functional food may have the active ingredients being added in an amount of about 15% by weight or less, more specifically about 10% by weight or less based on the total weight of the food or beverage raw materials. However, in the case of long-term intake for the purpose of health and hygiene or for the purpose of health management, the amount may be below the range.

The health functional food may further include at least one selected from carriers, diluents, excipients, or additives and then be formulated into one selected from the group consisting of tablets, pills, powders, granules, powders, capsules, and liquid formulations. Foods, to which compounds can be added according to one aspect, include various foods, powders, granules, tablets, capsules, syrups, beverages, gum, tea, vitamin complexes, and health functional foods.

Specific examples of the carriers, excipients, diluents, and additives include at least one selected from the group consisting of lactose, dextrose, sucrose, sorbitol, mannitol, erythritol, starch, gum acacia, calcium phosphate, alginate, gelatin, calcium phosphate, calcium silicate, microcrystalline cellulose, polyvinylpyrrolidone, cellulose, polyvinylpyrrolidone, methylcellulose, water, sugar syrup, methylcellulose, methyl hydroxy benzoate, propylhydroxy benzoate, talc, magnesium stearate, and mineral oil.

In addition to containing the effective ingredients, the health functional food may contain other ingredients as an essential ingredient without any particular limitations. For example, like regular beverages, the health functional food may contain various flavoring agents or natural carbohydrates as an additional ingredient. The examples of the natural carbohydrates mentioned above may be common sugars such as monosaccharides (for example, glucose and fructose); disaccharides (for example, maltose and sucrose); and polysaccharides (for example, dextrin and cyclodextrin) and sugar alcohols (for example, xylitol, sorbitol, and erythritol). As a flavoring agent other than those mentioned above, natural flavoring agents (thaumatin, stevia extract (for example, rebaudioside A and glycyrrhizin)) and synthetic flavoring agents (saccharin and aspartame) can be beneficially used. The ratio of the natural carbohydrates can be appropriately determined by the selection of those skilled in the art.

In addition, the health functional food according to one aspect may contain various nutrients, vitamins, minerals (electrolytes), flavoring agents such as synthetic and natural flavors, colorants and thickening agents (cheese and chocolate), pectic acids and the salts of the pectic acids, alginic acids and the salts of the alginic acids, organic acids, protective colloids thickening agents, pH adjusters, stabilizers, preservatives, glycerin, alcohol, and carbonating agents used in carbonated drinks. These ingredients can be used independently or in combination, and the ratio of these additives can also be appropriately selected by those skilled in the art.

In one embodiment of the present disclosure, the miRNA-214-3p inhibitor acts on phagocytosis, neuroinflammation, autophagy, RNA transport, or apoptosis caused by regulating the mRNA expression of the binding partner of the miRNA-214-3p, thereby the inhibitor enables to show the preventive or therapeutic effect of neurodegenerative diseases.

Additionally, the present disclosure provides the pharmaceutical agent for preventing or treating neurodegenerative diseases, the agent containing the miRNA-214-3p inhibitor as an active ingredient.

In one embodiment of the present disclosure, the pharmaceutical formulation may be in the form of an injectable formulation, an infusion formulation, a spray formulation, or a liquid formulation.

In addition, the present disclosure provides the method of preventing or treating neurodegenerative diseases, the method including administering a pharmaceutical composition containing the miRNA-214-3p inhibitor to an individual.

In the present disclosure, “individual” refers to a subject in need of treatment for diseases, and more specifically means mammals such as human or non-human primates, mice, rats, dogs, cats, horses, and cows.

Additionally, the present disclosure provides the use of the miRNA-214-3p inhibitor for preventing or treating neurodegenerative diseases.

Additionally, the present disclosure provides the use of the miRNA-214-3p inhibitor for use in manufacturing medicaments for preventing or treating neurodegenerative diseases.

Additionally, the present disclosure provides the use of the composition containing the miRNA-214-3p inhibitor for preventing or treating neurodegenerative diseases.

Additionally, the present disclosure provides the use of the composition containing the miRNA-214-3p inhibitor in manufacturing medicaments for use for preventing or treating neurodegenerative diseases.

Each of the features described herein can be used in combination, and the fact that each of the features is described in different dependent claims of the patent claims does not indicate that the features cannot be used in combination.

### Advantageous Effects

The present disclosure can be used for preventing and treating neurodegenerative diseases by using an inhibitor (inhibitor, siRNAs, shRNAs, or ASOs) of the miRNA-214-3p which is a miRNA that can regulate mRNAs involved in developing neurodegenerative diseases.

However, the effects of the present disclosure are not limited to the effects and should be understood to include all effects that can be inferred from the configuration of the disclosure described in the detailed description or claims of the present disclosure.

### DESCRIPTION OF DRAWINGS

FIG. 1 shows the results confirming the recovery of the decreased phagocytic capacity of iMGs derived from patients with ALS depending on treatment with a miRNA-214-3p inhibitor;

FIG. 2 shows the results confirming the decrease in mRNA expression of neuroinflammation-related factors depending on treatment with the miRNA-214-3p inhibitor;

FIG. 3 shows the results confirming the mRNA expression of autophagy-related factors depending on treatment with the miRNA-214-3p inhibitor;

FIG. 4 shows the results confirming the mRNA expression of Nucleocytoplasmic Trafficking-related factors depending on treatment with the miRNA-214-3p inhibitor; and

FIG. 5 shows the results confirming the recovery of the decreased phagocytic capacity of iMGs in patients with Alzheimer's disease depending on treatment with the miRNA-214-3p inhibitor.

### Mode for Disclosure

Hereinafter, the present disclosure will be described in more detail through examples. However, these examples are for illustrative purposes only and the scope of the present disclosure is not limited to these examples.

### EXAMPLES AND EXPERIMENTAL EXAMPLES

### Experimental Example 1. Method and Materials

### 1-1. Patient Recruitment and Clinical Protocol Approval

A trial was conducted with approval from the IRB of Hanyang University Hospital for patients with ALS and AD who meet diagnostic criteria.

1-2. Modeling of Induced Microglia (iMGs) Derived from Patients

Peripheral blood mononuclear cells (PBMC) were isolated through density gradient centrifugation using Ficoll, and then the PBMC were incubated under standard culture conditions (37° C., 5% CO₂) in RPMI-1640 media containing 10% of FBS and 1% of antibiotic/antimycotic solution. The next day, adherent cells (monocytes) were incubated for 21 days in RPMI-1640 Glutamax supplemented with 1% of antibiotic/antimycotic solution, 10 ng/ml of recombinant human granulocyte-macrophage colony-stimulating factor (GM-CSF), and 100 ng/ml of recombinant human interleukin-34 (IL-34), thereby modeling iMGs.

1-3. miRNA Inhibitor Transfection

A miR-214 mimic or a miR-214 inhibitor, and a control group of oligonucleotides were synthesized by Bioneer Corporation (Daejeon, Korea). The synthesized miRNA mimic, inhibitor, and negative control group were transfected into iMGs using Lipofectamine® RNAiMAX (Invitrogen) according to the manufacturer's instructions. Specifically, 50 nM of the miRNA mimic, miRNA inhibitor, or negative control group each was transfected into cells plated at a density of 3×10⁵ cells/well in a 6-well plate (see Table 1).

TABLE 1

| miRNA | Sequence |
|---|---|
| miRNA-214-3p mimic | 5′-UGCCUGUCUACACUUGCUGUGC-3′ (SEQ ID NO: 1) |
| miRNA mimic negative | Bioneer Corporation, Korea, #SMC-2003 |
| control #1 | |
| miRNA-214-3p inhibitor | 5′-ACAGCAGGCACAGACAGGCAGU-3′ (SEQ ID NO: 2) |
| miRNA inhibitor negative | Bioneer Corporation, Korea, #SMC-2103 |
| control #1 | |

### 1-4. Phagocytosis Assay

iMGs were treated with 4 μl of red fluorescent latex beads at a temperature of 37° C. for 4 hours. The cells were washed twice with ice-cold PBS, fixed, and stained with F-actin and DAPI. Images were taken by using a confocal microscope (TCS SP5 produced by Leica, Wetzlar, Germany). The number of phagocytosed beads per cell was calculated using Image J software for phagocytic activity.

1-5. Analysis of Quantitative Real Time-Polymerase Chain Reaction (qRT-PCR)

Total RNAs were extracted using Trizol reagent (Invitrogen) and evaluated using a NanoDrop 2000 spectrophotometer (Thermo Scientific, ND-2000). cDNAs were synthesized using an EcoDry™ CDNA kit (Clontech, CA, USA). The cDNAs were amplified using a Power SYBR Green PCR Master Mix with primers in an Applied Biosystems Step One Plus™ system (Life Technologies) at a temperature of 95° C. for 10 minutes. Afterward, the process of amplification with the cDNAs at a temperature of 95° C. for 15 seconds and then at a temperature of 60° C. for 1 minute was set as one cycle and the cycle was repeated 40 times. A melting curve was produced to analyze the specificity of the amplification. A relative quantity (RQ) level was calculated based on the 2-ΔΔCt method using GAPDH as an internal standard control group. The reported results are based on three independent trials performed with separate batches of the cells. Used primers were NCKAP1 (Qiagen, PPH15666A), WASF2 (PPH01258A), ABL2 (PPH00066E), TRAF1 (PPH00815B), TNFSF10 (PPH00242F), IKBKB (PPH00780C), RUBCN (PPH22660B), ATG16L1 (PPH19850A), ATG12 (PPH15326A), TNPO1 (PPH19196A), IPO11 (PPH20990A), KPNA1 (PPH13213B), and GAPDH (Qiagen, PPH00150F).

### 1-6. Enzyme-Linked Immunosorbent Assay

Secretion of inflammatory cytokines (TNF-α, IL-1β) obtained from culture supernatants was analyzed using a commercially available cytokine assay kit obtained from Millipore (Billerica, MA) in accordance with the manufacturer's protocol.

### 1-7. Statistical Analysis

Data were expressed as mean±SEM. The statistical significance of differences between groups was assessed through t-test and one-way ANOVA with a Tukey's post hoc test using Prism 9 (GraphPad Software, San Diego, CA).

### Experimental Example 2. Results

### 2-1. Confirmation of Phagocytic Capacity Recovery

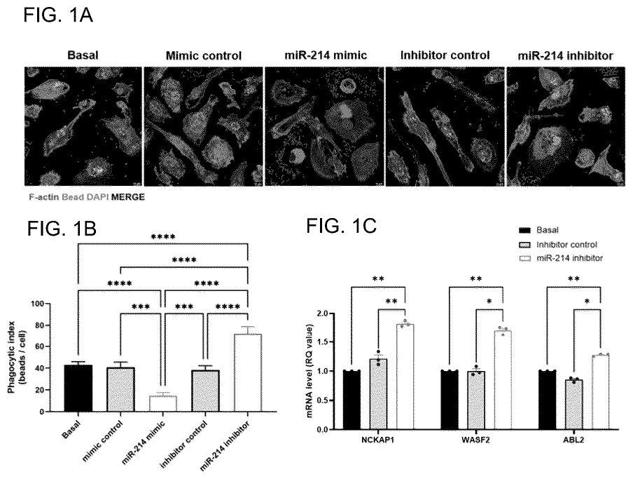

To confirm the effect of the miRNA-214-3p inhibitor in the iMGs (induced microglia) having decreased phagocytic capacity and derived from patients with ALS, RNAis were used for transfection with basal (mock), mimic n.c (negative control), miR-214 mimic, inhibitor n.c, and miR-214 inhibitor. After the transfection, cell immunostaining was performed to evaluate bead phagocytic capacity (FIG. 1A). The results were quantified and graphed (FIG. 1B). In addition, changes in the mRNA expression of NCKAP1, WASF2, and ABL2 were confirmed depending on treatment with the miRNA-214-3p inhibitor. For reference, NCKAP1, WASF2, and ABL2 were a regulator of actin cytoskeleton, and the actin cytoskeleton played a key role in the phagocytic capacity while the miRNA-214 targeted the actin cytoskeleton dynamics (FIG. 1C).

As a result, all the expression of NCKAP1, WASF2, and ABL2 miRNAs was confirmed to increase upon administration of the miRNA-214-3p inhibitor. This indicated that administration of the miRNA-214-3p inhibitor had preventive and therapeutic effects on neurodegenerative diseases.

2-2. Confirmation of mRNA Expression of Factors Related to Neuroinflammation

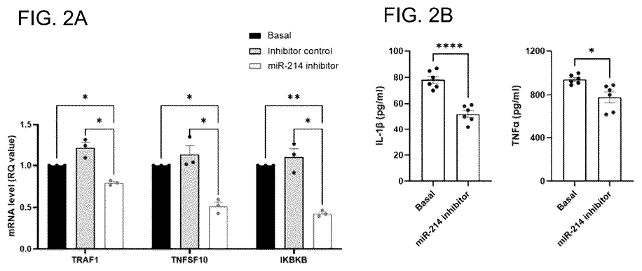

After treatment with the miRNA-214-3p inhibitors, the mRNA expression of TRAF1, TNFSF10, and IKBKB was confirmed as one of the main mechanisms of neurodegenerative diseases, the TRAF1, TNFSF10, and IKBKB mRNAs affecting the TNF/NFKB pathway as a key factor in neuroinflammation and being the target of the miRNA-214 (FIG. 2A). In addition, after treatment with the miRNA-214-3p inhibitors, a decrease in neuroinflammatory factors (TNFa, IL-1beta) secreted from the culture fluid of the iMGs derived from patients with ALS was confirmed (FIG. 2B).

As a result, all the mRNA expression of TRAF1, TNFSF10, and IKBKB was confirmed to decrease when the miRNA-214-3p inhibitor was administered. This indicated that administration of the miRNA-214-3p inhibitor had preventive and therapeutic effects on neurodegenerative diseases.

2-3. Confirmation of mRNA Expression of Autophagy-Related Factors

After treatment with the miRNA-214-3p inhibitor, changes in the mRNA expression of RUBCN, ATG16L1, and ATG12 were confirmed as one of the main mechanisms of the neurodegenerative diseases, the RUBCN, ATG16L1, and ATG12 mRNAs being autophagy-related factors and being the target of miRNA-214.

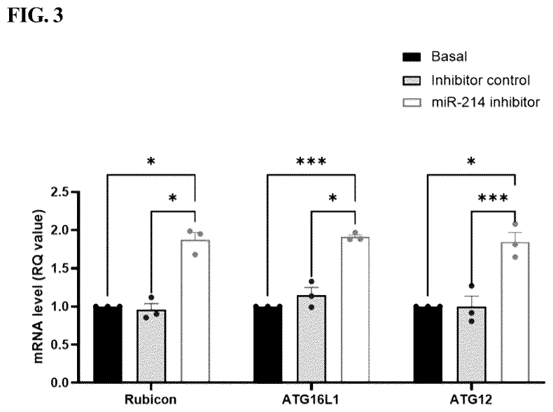

As a result, the mRNA expression of RUBCN, ATG16L1, and ATG12 was confirmed to increase when treated with the inhibitor compared to the control group (FIG. 3). This indicated that administration of the miRNA-214-3p inhibitor had preventive and therapeutic effects on neurodegenerative diseases.

2-4. Confirmation of mRNA Expression of Factors Related to Nucleocytoplasmic Trafficking

After treatment with the miRNA-214-3p inhibitor, changes in the mRNA expression of TNPO1, IPO11, and KPNA1 were confirmed as one of the main mechanisms of the neurodegenerative diseases, the TNPO1, IPO11, and KPNA1 mRNAs being factors related to Nucleocytoplasmic Trafficking and being the target of miRNA-214.

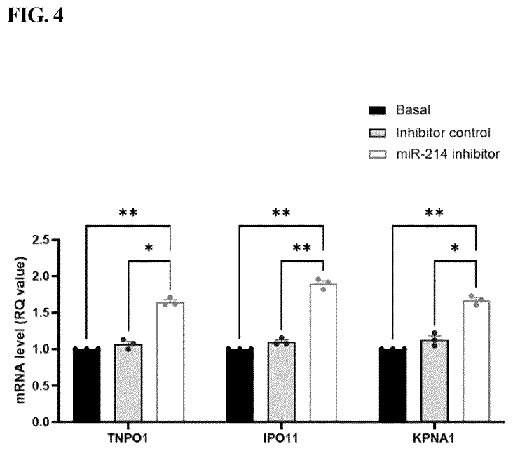

As a result, the mRNA expression of TNPO1, IPO11, and KPNA1 was confirmed to increase when treated with the inhibitor compared to the control group (FIG. 4). This indicated that administration of the miRNA-214-3p inhibitor had preventive and therapeutic effects on neurodegenerative diseases.

2-5. Confirmation of Recovery of Decreased Microglial Phagocytic Capacity in iMGs Derived from Patients with Alzheimer's Disease

To confirm the effect of the miRNA-214-3p inhibitor in the iMGs (induced microglia) having decreased phagocytic capacity and derived from patients with Alzheimer's disease (with the severity of moderate to late AD), RNAis were used for transfection with basal (mock), mimic n.c, miR-214 mimic, inhibitor n.c, and miR-214 inhibitor. After the transfection, bead phagocytic capacity was evaluated.

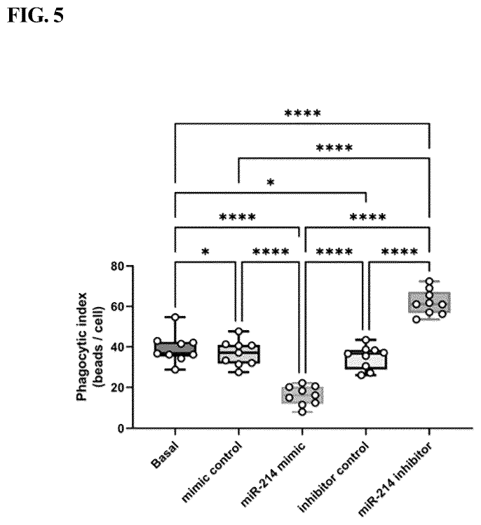

As a result, the decreased microglial phagocytic capacity was confirmed to be restored in the administration case of the miRNA-214-3p inhibitor (FIG. 5). This indicated that administration of the miRNA-214-3p inhibitor had preventive and therapeutic effects on neurodegenerative diseases.

1. A pharmaceutical composition for preventing or treating a neurodegenerative disease, the composition comprising a miRNA-214-3p inhibitor as an active ingredient.

2. The pharmaceutical composition of claim 1, wherein the inhibitor is an activity inhibitor or expression inhibitor against miRNA-214-3p; or an activity inhibitor or expression inhibitor against a binding partner of the miRNA-214-3p.

3. The pharmaceutical composition of claim 2, wherein the activity inhibitor is at least one selected from the group consisting of a compound, a peptide, a peptide mimic, a substrate analog, an aptamer and an antibody, binding specifically to the miRNA-214-3p or a binding partner protein of the miRNA-214-3p.

4. The pharmaceutical composition of claim 2, wherein the expression inhibitor is at least one selected from the group consisting of antisense oligonucleotides, RNAi, siRNA, miRNA, shRNA and ribozyme, binding complementarily to the miRNA-214-3p or to an mRNA of a binding partner gene to miRNA-214.

5. The pharmaceutical composition of claim 1, wherein the miRNA-214-3p inhibitor is represented by the base sequence of SEQ ID NO: 3.

6. The pharmaceutical composition of claim 1, wherein the neurodegenerative disease is selected from the group consisting of Parkinson's disease, dementia, Alzheimer's disease, frontotemporal dementia, Huntington's disease, and ALS.

7. A health functional food composition for preventing or improving a neurodegenerative disease, the health functional food composition comprising a miRNA-214-3p inhibitor as an active ingredient.

8. A pharmaceutical preparation for preventing or treating a neurodegenerative disease, the pharmaceutical preparation comprising a miRNA-214-3p inhibitor as an active ingredient.

9. A method of preventing or treating a neurodegenerative disease, the method comprising administering to a subject a pharmaceutical composition containing a miRNA-214-3p inhibitor.

10. A use of a miRNA-214-3p inhibitor for use in manufacturing for preventing or treating a neurodegenerative disease.
